(19) United States
(12) Patent Application Publication
(10) Pub. No.: US 2006/0292663 A1
(43) Pub. Date: Dec. 28, 2006

(54) Method of measuring lactate dehydrogenase activity in serum, and device for measuring lactate dehydrogenase activity in serum

(75) Inventors: Hidenobu Yaku, Osaka (JP); Tetsuo Yukimasa, Osaka (JP)

Correspondence Address: McDERMOTT WILL & EMERY LLP, 600 13th Street, N.W., Washington, DC, 20005-3096, US

(73) Assignee: MATSUSHITA ELECTRIC INDUSTRIAL CO. LTD., Osaka (JP)

(21) Appl. No.: 11/498,128

(22) Filed: Aug. 3, 2006

Related U.S. Application Data
(63) Continuation of application No. PCT/JP06/11250, filed on Jun. 5, 2006.

(30) Foreign Application Priority Data
Jun. 28, 2005 (JP) 2005-188696

(51) Int. Cl. C12Q 1/30

(52) U.S. Cl. 435/27

(57) ABSTRACT

A new electrochemical measurement method is provided that is excellent in accuracy in detecting LDH activity in a serum sample and shortens the time required for the detection. The method of measuring lactate dehydrogenase activity in serum includes: a first step (S1) of adding at least lactic acid, a coenzyme of a lactate dehydrogenase, and an electron mediator to a first serum sample that contains the lactate dehydrogenase to prepare a second serum sample; a second step (S2) of removing molecules whose molecular weights are 30000 or higher, preferably molecules whose molecular weights are 10000 or higher from the second serum sample to prepare a third serum sample; and a third step (S3) of measuring a value of current that is generated by applying voltage to the third serum sample.

### BACKGROUND OF THE INVENTION

1. Field of the Invention

The present invention relates to a method of electrochemically measuring the activity of lactate dehydrogenase (LDH) in serum. Furthermore, the present invention also relates to a measurement device that is suitable for the measurement described above.

2. Related Background Art

It is considered that analyzing qualitatively and quantitatively the concentration and activity of biomolecules (protein, low molecules, sugar, nucleic acid, etc.) in biological samples such as blood, urine, cells, etc. is highly important in clinical and healthcare fields, such as prevention of diseases, illness diagnosis, and healthcare administration.

For example, it has been know that although glutamic-oxaloacetic transaminase (GOT) and glutamic-pyruvic transaminase (GPT) are enzymes that are retained in hepatocyte, they leak into blood when an abnormality such as damages to hepatocyte occurs. It therefore is considered that diagnostic indices of chronic hepatitis, hepato cirrhosis, fatty liver, etc., can be obtained by measuring the activity of the above-mentioned enzymes in blood, for example. Furthermore, the glucose concentration in blood may show abnormal values due to pancreatitis, thyroid disease, dumping syndrome after gastrectomy, etc. and furthermore due to obesity, lack of exercise, stress, etc. Accordingly, it is receiving attention as a diagnostic index that has great versatility.

The lactate dehydrogenase (hereinafter also referred to as “LDH” in some cases) in blood is receiving attention as biomolecules that can exhibit greater versatility as a diagnostic index of damages to biological tissues, internal organs, etc. Reasons for this include the fact that LDH exists in almost all tissues and internal organs in vivo. Furthermore, another reason is the fact that like the GOT and GPT, LDH leaks into blood from damaged tissues or internal organs, which causes an increase in the LDH activity in blood. LDH is an enzyme that catalyzes the conversion reaction between pyruvic acid and lactic acid.

Conventionally, an optical detection method that employs a fluorescent marker, etc. is used for tests and analyses of such biomolecules that have great values in clinical and healthcare fields. In such an optical technique, an illuminant, a fluorescent indicator, etc. are indispensable. For this reason, such an optical technique has problems in that the size of the measurement device is difficult to reduce and the costs of the measurement device and measurement method itself are high. Hence, there are great demands for development of the measurement technology using an electrochemical technique that relatively facilitates the reductions in size of the device and in costs of the device and measurement method.

For the purpose of electrochemically measuring the LDH activity in blood, techniques have been proposed that utilize redox reactions indicated by the chemical formulae 1 to 3 described below (see patent documents: JP54(1979)-139792A, JP54(1979)-139793A, JP56(1981)-35051A, JP59(1984)-98681A, and JP2000-224999A). In such techniques, voltage is applied to serum using a platinum electrode, an electrode that is produced by mixing nicotinamide adenine dinucleotide (hereinafter also referred to as “NAD” in some cases) and a collector, or an electrode that is produced by fixing NAD on a collector by chemical bonds, and thereby current is detected that is generated depending on the LDH activity in blood.

The redox reactions are described below. First, lactic acid and NAD are added to the sample to be subjected to the measurement. At this stage, if the sample contains LDH, the reaction that is expressed by the following chemical formula 1 progresses and thereby pyruvic acid and reduced nicotinamide adenine dinucleotide (hereafter also referred to as “NADH” in some cases) are produced in an amount corresponding to the activity (concentration) of LDH that is contained in the sample.

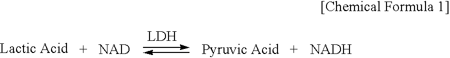

Subsequently, a catalyst such as, for example, diaphorase and an oxidized electron mediator such as, for example, potassium hexacyanoferrate (III) (potassium ferricyanide) are added to the sample. Then the reaction that is indicated by the following chemical formula 2 progresses and the electron mediator is converted from an oxidized form into a reduced form according to the amount of NADH in the sample.

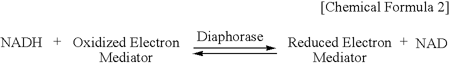

Thereafter, when a predetermined voltage is applied to the sample, the reduced electron mediator is reconverted into the oxidized electron mediator as is indicated in the following chemical formula 3. Due to this reconversion, oxidation current (hereinafter also referred to as “response current” in some cases) corresponding to the amount of reduced electron mediator is generated in the sample.
[Chemical Formula 3]

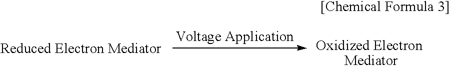

### SUMMARY OF THE INVENTION

However, as a result of the studies made by the present inventors, the following problem was found. That is, in the techniques described in the above-mentioned patent application publication, the response current that is obtained by applying constant voltage is small and the stability thereof is low. Therefore it is not easy to measure the LDH concentration in a short time with high accuracy.

The mechanism that causes such deterioration in measurement accuracy and such an impediment to shortening the measurement time is unclear in many respects. However, one of the factors is considered as follows. That is, proteins that exist in serum adsorb to the surfaces of the measurement electrodes and thereby reduce the effective areas of the measurement electrodes. Since at least 100 kinds of proteins exist in blood, it is not easy to specify the responsible protein that causes the deterioration in measurement accuracy. However, according to the general common knowledge in the technical field concerned, albumin and various types of immunoglobulins (alpha 1, alpha2, beta, gamma, etc.) are predicted as the candidates. This is because such proteins each have a high mass ratio in serum and have a dominant effect on variations in the total amount of proteins in serum.

Hence, it is predicted that the above-mentioned problems can be solved by removing both albumin (whose molecular weight is approximately 66000) and immunoglobulin (whose molecular weight is 150000 to 900000) in a serum sample. However, contrary to such a prediction based on the general common knowledge in the technical field concerned, the present inventors have found out that even if filtering for removing molecules with molecular weights of 50000 or higher that were contained in a serum sample, i.e. filtering that allowed both albumin and immunoglobulin to be removed, was carried out, the LDH concentration was not able to be measured in a short time with high accuracy.

The present invention therefore is intended to provide a new electrochemical measurement method that is excellent in accuracy in detecting the LDH activity in a serum sample and that shortens the time required for the detection. Furthermore, the present invention is intended to provide a measurement device that is suitable for the above-mentioned new measurement method.

As a result of keen studies made assiduously in order to solve the above-mentioned problems, the present inventors have found out the following. The treatment for removing molecules with molecular weights of 50000 or higher from a serum sample before the LDH activity was measured electrochemically was not sufficient. Surprisingly, it was not until biomolecules with molecular weights of 30000 or higher were removed that the signal strength and stability of response current improved. Through these findings, the present invention was completed.

The present invention provides a method of measuring lactate dehydrogenase activity in serum. The method includes: a first step of adding at least lactic acid, a coenzyme of a lactate dehydrogenase, and an electron mediator to a first serum sample that contains the lactate dehydrogenase to prepare a second serum sample; a second step of removing molecules whose molecular weights are 30000 or higher from the second serum sample to prepare a third serum sample after the first step; and a third step of measuring a value of current that is generated by applying voltage to the third serum sample.

Furthermore, the present invention provides a device for measuring lactate dehydrogenase activity in serum as a measurement device that is suitable for carrying out the above-mentioned measurement method. The measurement device includes: a sample inlet for feeding a serum sample that contains lactate dehydrogenase; a reaction bath for mixing the serum sample, lactic acid, a coenzyme of the lactate dehydrogenase, and an electron mediator, with the reaction bath being in communication with the sample inlet; a first electrode bath provided with electrodes; and a molecule exclusion channel that allows the reaction bath and the first electrode bath to be in communication with each other. The molecule exclusion channel is provided with a member for removing molecules whose molecular weights are 30000 or higher from the serum sample that is discharged from the reaction bath.

The present invention provides a new electrochemical measurement method that has improved accuracy in detecting the LDH activity in a serum sample and that can shorten the time required for the detection. Furthermore, the present invention can provide a measurement device that is suitable for the measurement.

### BRIEF DESCRIPTION OF THE DRAWINGS

FIG. 1A is a graph showing change in detected current with time in the constant-potential measurement according to a measurement method of Comparative Example 1.

FIG. 1B is a graph showing change in detected current with time in the constant-potential measurement according to a measurement method of Comparative Example 2.

FIG. 1C is a graph showing change in detected current with time in the constant-potential measurement according to a measurement method of Example 1.

FIG. 1D is a graph showing change in detected current with time in the constant-potential measurement according to a measurement method of Example 2.

FIG. 2 is a graph showing, in a comparative manner, peak values of the detected currents in the constant-potential measurement, which were obtained by the measurement methods according to the examples and comparative examples.

FIG. 3 is a flow chart that is used for explaining an example of the measurement method of the present invention.

FIG. 4 is a flow chart that is used for explaining another example of the measurement method of the present invention.

FIG. 5 is a diagram that is used for explaining an example of the measurement device of the present invention.

FIG. 6 is a diagram that is used for explaining another example of the measurement device of the present invention.

FIG. 7 is a graph showing an example of the relationship between LDH activity and detected current values that were obtained 180 seconds after the initiation of constant-potential measurement.

FIG. 8 is a graph showing an example of the relationship between LDH activity and detected current values that were obtained 300 seconds after the initiation of constant-potential measurement.

FIG. 9 is a graph showing another example of the relationship between LDH activity and detected current values that were obtained 180 seconds after the initiation of constant-potential measurement.

FIG. 10 is a graph showing another example of the relationship between LDH activity and detected current values that were obtained 300 seconds after the initiation of constant-potential measurement.

### DETAILED DESCRIPTION OF THE INVENTION

### Embodiment 1

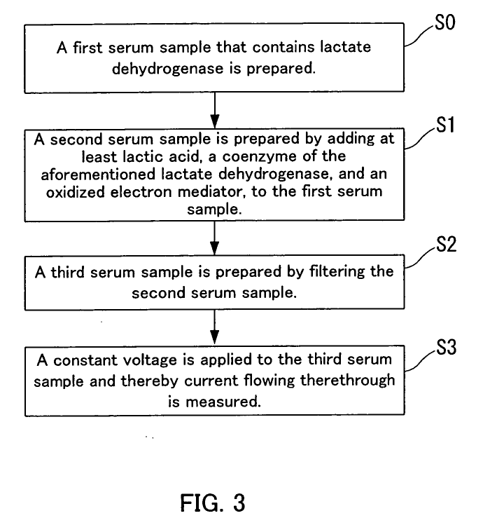

A method of measuring lactate dehydrogenase activity in serum according to Embodiment 1 of the present invention is described with reference to the flow chart shown in FIG. 3.

[Step S0: Preparation of First Serum Sample]

First, a first serum sample containing lactate dehydrogenase is prepared. For example, when the sample is whole blood, blood cells are separated therefrom using a well-known means.

[Step S1: Preparation of Second Serum Sample]

Next, at least lactic acid, a coenzyme of the aforementioned lactate dehydrogenase, and an electron mediator are added to the first serum sample. Thus a second serum sample is prepared.

The above-mentioned coenzyme is not particularly limited as long as it assists the activity of lactate dehydrogenase. For instance, at least one selected from nicotinamide adenine dinucleotide (NAD), nicotinamide adenine dinucleotide phosphate (NADP), etc. can be used.

The electron mediator to be used herein can be of an oxidized type. From the viewpoints of improvements in solubility in a serum sample, the efficiency of transferring electrons between measurement electrodes and itself, etc., it is preferable that the electron mediator to be used herein be, for example, ferricyanide(hexacyanoferrate(III) (potassium, sodium)), 1,2-naphthoquinone-4-sulfonate(potassium, sodium), 2,6-dichlorophenol indophenol, dimethylbenzoquinone, 1-methoxy-5-methylphenazinium methyl sulfate, methylene blue, gallocyanine, thionine, phenazine methosulfate, Meldola's blue, etc. Among them, it is preferable that potassium hexacyanoferrate(III) be used.

Preferably, diaphorase further is added to the second serum sample. This is because when diaphorase catalyzes the reaction between the electron mediator and the coenzyme, the time required for preparing the second serum sample may be shortened in some cases.

This step S1 allows a redox reaction to progress between the coenzyme and electron mediator in the second serum sample. For example, when an oxidized electron mediator is added to the first serum sample, the electron mediator is converted into a reduced type through the step S1. As is shown by the chemical formula 2 indicating the redox reaction, the amount of electron mediator to be converted depends on the activity (concentration) of LDH that is contained in the first serum sample.

[Step S2: Preparation of Third Serum Sample]

Subsequently, the second serum sample is filtered under a first filtering condition where molecules with molecular weights of 30000 or higher are removed, preferably a second filtering condition where molecules with molecular weights of 10000 or higher are removed. Thus a third serum sample is prepared. Although it will be described later in detail, it is important to remove even biomolecules with molecular weights of 30000 or higher (preferably 10000 or higher). Based on the general common knowledge in the technical field concerned, it is predicted that the filtering condition to be employed herein may be a condition where both albumin and immunoglobulin are removed from the serum sample, i.e. a condition where molecules with molecular weights of 50000 or higher are removed from the serum sample. Such a treatment condition, however, is not sufficient.

The method for removing molecules with the above-mentioned molecular weights from serum can be a well-known method such as a method in which for example, a filter is prepared that has a mesh structure with a pore size according to the objects to be removed, and serum is filtered through the filter.

[Step S3: Constant-Potential Measurement]

Next, voltage is applied to the third serum sample described above. This allows the reduced electron mediator contained in the third serum sample to be converted into an oxidized electron mediator, for example. Then the value of current that is generated through the conversion is measured. In this case, the voltage to be applied is equal to or higher than oxidation potential of the reduced electron mediator.

The amount of the current that is generated through the conversion depends mainly on the activity of LDH contained in the first serum sample as is shown by the chemical formulae 1 to 3 indicating the redox reactions. Accordingly, the detection of this current amount allows the activity of LDH in serum to be measured electrochemically.

Furthermore, although the detail is described later, when using the method of measuring lactate dehydrogenase activity in serum that includes the steps S1 to S3, response current that reaches a steady state can be obtained between 180 seconds and 600 seconds after the initiation of the voltage application when the above-mentioned first filtering condition is employed, and between 80 seconds and 600 seconds when the above-mentioned second filtering condition is employed. It also is possible to increase the magnitude of signals of the response current considerably or to obtain response current that reflects the magnitude of LDH activity. This makes it possible not only to improve the accuracy in detecting the LDH activity in serum but also to shorten the time required for the detection. As described later, even if the filtering is carried out under the condition where both albumin and immunoglobulin are removed from the serum sample, it is not possible to improve the accuracy in detecting the LDH activity in serum. This finding made by the present inventor is unexpected according to the general common knowledge in the technical field concerned. Hence, at the present stage, it is not clear why it is only after the removal of molecules with molecular weights of 30000 or higher that such an excellent effect is obtained.

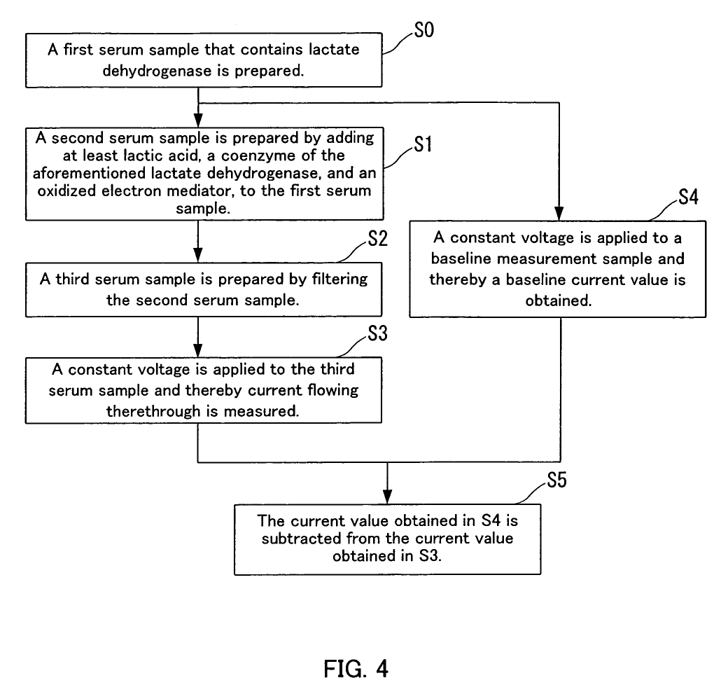

Preferably, the measurement method of the present invention further includes the following steps S4 and S5. This will be described with reference to the flow chart shown in FIG. 4.

[Step S4: Measurement of Baseline Current Value]

Using the same method as that employed in the step S2, molecules with molecular weights of 30000 or higher, preferably molecules with molecular weights of 10000 or higher are removed from the first serum sample, without adding the compounds to be added in the step S1, such as lactic acid, a coenzyme, an electron mediator, etc. Thus a baseline measurement sample is obtained. Voltage is applied to the baseline measurement sample in the same manner as in the step S3. Then the value of current that is generated in the baseline measurement sample is obtained as a baseline current value. This makes it possible to detect the response current (noise) in the background that is generated due to an intrinsic electron mediator that can be present in the first serum sample. This step S4 may be carried out before the completion of the step S3 or may be carried out after the step S3.

[Step S5: Correction of Detected Current Value]

The baseline current value obtained in the step S4 is subtracted from the current value obtained in the step S3. This allows noise current in the background to be removed and thereby the LDH activity in the serum sample subjected to the measurement can be measured with higher accuracy. The respective current values to be used for the subtraction are detected at the same point in time after the initiation of the voltage application. For instance, they can be detected 80 seconds, 180 seconds, and 300 seconds after the initiation of the voltage application. It is preferable that the serum sample to be used in the step S3 and the baseline measurement sample to be used in the step S4 have been treated under the same filtering conditions.

The method of measuring lactate dehydrogenase activity in serum including the above-mentioned steps S1 to S5 allows the LDH activity in serum to be measured with further higher accuracy.

### Embodiment 2

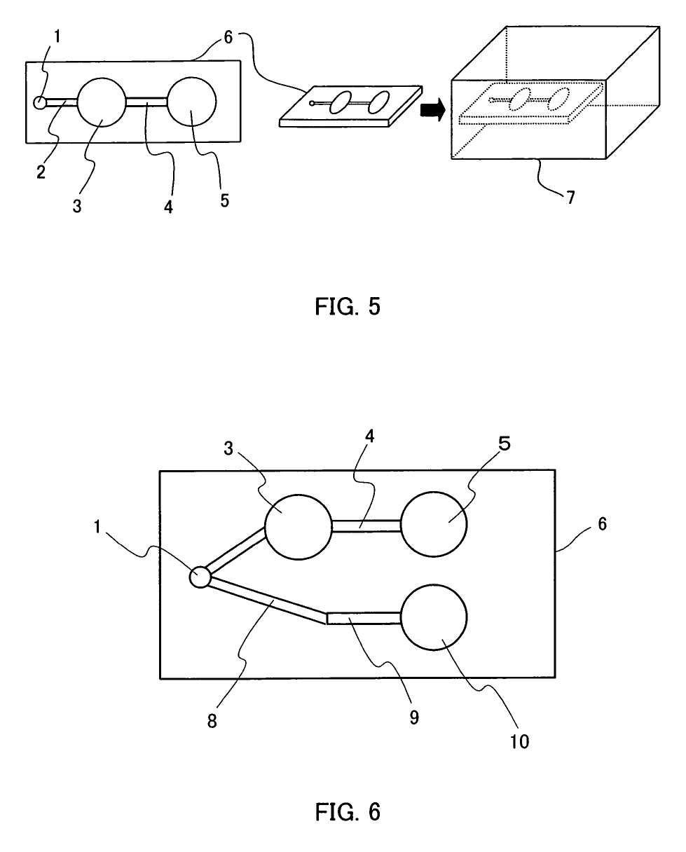

A device for measuring lactate dehydrogenase activity in serum according to Embodiment 2 of the present invention is described with reference to FIG. 5. As shown in FIG. 5, this measurement device 7 is provided with a reaction vessel 6. The reaction vessel 6 includes: a sample inlet 1; a reaction bath 3 that is in communication with the sample inlet 1; a first electrode bath 5; and a molecule exclusion channel 4 that allows the reaction bath 3 and the first electrode bath 5 to be in communication with each other.

The sample inlet 1 is an inlet for feeding a serum sample (a first serum sample) that contains lactate dehydrogenase, into the reaction vessel 6.

A channel 2 may be provided between the sample inlet 1 and the reaction bath 3 so as to allow them to be in communication with each other as shown in FIG. 5. On the other hand, the sample inlet 1 may be provided directly for a portion of the reaction bath 3, without providing the channel 2, so that they may be allowed to be in communication with each other.

The reaction bath 3 is used for mixing a serum sample, lactic acid, a coenzyme of lactate dehydrogenase, and an electron mediator. In this reaction bath, the step S1 of Embodiment 1 can be carried out. With respect to the above-mentioned mixing to be carried out in the reaction bath, the following manners may be employed: the compounds to be added in the step S1, for example, lactic acid, a coenzyme, an electron mediator, etc. may have been retained in the reaction bath beforehand, or such compounds may be fed through the sample inlet 1 at the time of measurement.

The molecule exclusion channel 4 is provided with a first filtering member, preferably a second filtering member. The first filtering member removes molecules with molecular weights of 30000 or higher from the serum sample discharged from the reaction bath 3. The second filtering member removes molecules with molecular weights of 10000 or higher from the serum sample. The serum sample is sent from the reaction bath 3 to the first electrode bath 5 through the molecule exclusion channel, so that the step S2 of Embodiment 1 can be carried out.

The serum sample can be sent from the reaction bath 3 to the first electrode bath 5 by the following methods. For example, it can be carried out through pressurization using a pump, or with centrifugal force that is generated towards the first electrode bath from the reaction bath using a centrifuge. Similarly in the case where the channel 2 is provided between the sample inlet 1 and the reaction bath 3, the serum sample can be sent in the same manner as described above.

A well-known separation member may be provided between the sample inlet 1 and the reaction bath 3. The separation member allows blood cells and serum to be separated from each other to send the serum alone to the reaction bath. In this case, it is not necessary to prepare serum as a measurement sample. The LDH activity can be measured by simply preparing whole blood.

The first electrode bath 5 is provided with electrodes for carrying out constant-potential measurement. The electrodes may be configured as a two electrode type that is composed of a working electrode and a counter electrode or a three electrode type that is composed of a working electrode, a counter electrode, and a reference electrode. Although it is not shown in FIG. 5, this measurement device 7 includes a means for applying a predetermined voltage to the electrodes provided for the first electrode bath 5 and a means for detecting the value of current that flows through the electrodes. In this first electrode bath, the step S3 of Embodiment 1 can be carried out.

For the purpose of removing background noise from the detected current, it is preferable that the above-mentioned reaction vessel 6 further include a second electrode bath 10 and a second molecule exclusion channel 9 that allows the sample inlet 1 and the second electrode bath 10 to be in communication with each other, in addition to the sample inlet 1, the reaction bath 3, the first electrode bath 5, and the molecule exclusion channel 4 as shown in FIG. 6. The second molecule exclusion channel 9 may be in direct connection with the sample inlet 1 or may be in communication therewith through a second channel 8 as shown in FIG. 6.

The second molecule exclusion channel 9 is provided with a first filtering member, preferably a second filtering member. The first filtering member removes molecules with molecular weights of 30000 or higher from the serum sample fed from the sample inlet 1. The second filtering member removes molecules with molecular weights of 10000 or higher from the serum sample. The serum sample is sent to the second electrode bath 10 through the second molecule exclusion channel, so that the baseline measurement sample according to Embodiment 1 can be prepared.

The serum sample can be sent to the second electrode bath 10 through the second molecule exclusion channel 9 by the following methods. For example, it can be carried out through pressurization using a pump, or with centrifugal force that is generated towards the first electrode bath from the reaction bath using a centrifuge. Similarly in the case where a second channel 8 is provided between the sample inlet 1 and the second molecule exclusion channel 9, the serum sample can be sent in the same manner as described above.

A well-known separation member may be provided for the second channel 8. The separation member allows blood cells and serum to be separated from each other to send the serum alone to the reaction bath. In this case, as described above, whole blood can be used as a measurement sample and therefore it is not necessary to prepare serum.

The second electrode bath 10 is provided with electrodes for carrying out constant-potential measurement as in the case of the first electrode bath 5 described above. In this second electrode bath, the step S4 of Embodiment 1 can be carried out. This measurement device 7 includes a means for applying a desired voltage to the electrodes provided for the second electrode bath and a means for detecting the value of current that flows through the electrodes.

Furthermore, although it is not shown in the drawings, this measurement device 7 is provided with a computing means that subtracts the value of current detected in the second electrode bath 10 from the value of current detected in the first electrode bath 5 through the constant-potential measurement. With this computing means, the step S5 of Embodiment 1 can be carried out.

### EXAMPLES

Hereinafter, the present invention is described further in detail using examples but is not limited thereto.

### Example 1

LDH activity in a serum sample was measured as described below. First, “Fluid Control Serum I Wako (Code 418-00401)” manufactured by Wako Pure Chemical Industries, Ltd. that contained 350 unit/liter (U/L) of LDH was prepared as a serum sample A, while “Fluid Control Serum II Wako (Code 414-00501)” manufactured by Wako Pure Chemical Industries, Ltd. that contained 772 U/L of LDH was prepared as a serum sample B.

Then, 50 mM of Tris-HCl buffer (ph 8.5), 1 mM of nicotinamide adenine dinucleotide (NAD), 1 mM of potassium ferricyanide, 10 mM of lithium lactate, and 12.5 U/L of diaphorase were added to each of the 100-microliter (μL) serum samples A and B. Thus 200-μL serum samples A2 and B2 were prepared. Subsequently, the serum samples A2 and B2 were incubated at 30° C. for five minutes.

After the incubation, each serum sample was filtered through a centrifugal filter (Amicon 30,000, manufactured by Amicon Inc.) that was able to remove molecules with molecular weights of 30000 or higher. Hereinafter, the respective serum samples obtained after the filtering are referred to as “serum samples A3 and B3”. The filtering was carried out by a centrifugal process in which a centrifugal force of 12000G was applied for three minutes.

Then 40 μL of each of the serum samples A3 and B3 was measured out and fed into an electrode bath for constant-potential measurement. The electrode bath was provided with a working electrode, a counter electrode, and a reference electrode. Thereafter, a voltage of 400 mV with reference to a silver/silver chloride electrode was applied to each of the serum samples A3 and B3 and thereby current generated in each serum sample was monitored up to 600 seconds from the initiation of the voltage application. This constant-potential measurement was carried out using a carbon electrode for each of the working electrode and the counter electrode and the silver/silver chloride electrode for the reference electrode.

### Example 2

Example 2 was an experimental example in which the constant-potential measurement was carried out in the same manner as in Example 1 except that the centrifugal filter used herein was a centrifugal filter (Amicon 10,000, manufactured by Amicon Inc.) that was able to remove molecules with molecular weights of 10000 or higher.

### Comparative Example 1

Comparative Example 1 is an experimental example in which the LHD activity in a serum sample was measured in the same manner as in Example 1 except that current was monitored by carrying out the constant-potential measurement without filtering each serum sample after the incubation.

### Comparative Example 2

Comparative Example 2 is an experimental example in which the LHD activity in a serum sample was measured in the same manner as in Example 1 except that the centrifugal filter used herein was a centrifugal filter (Amicon 50,000, manufactured by Amicon Inc.) that was able to remove molecules with molecular weights of 50000 or higher.

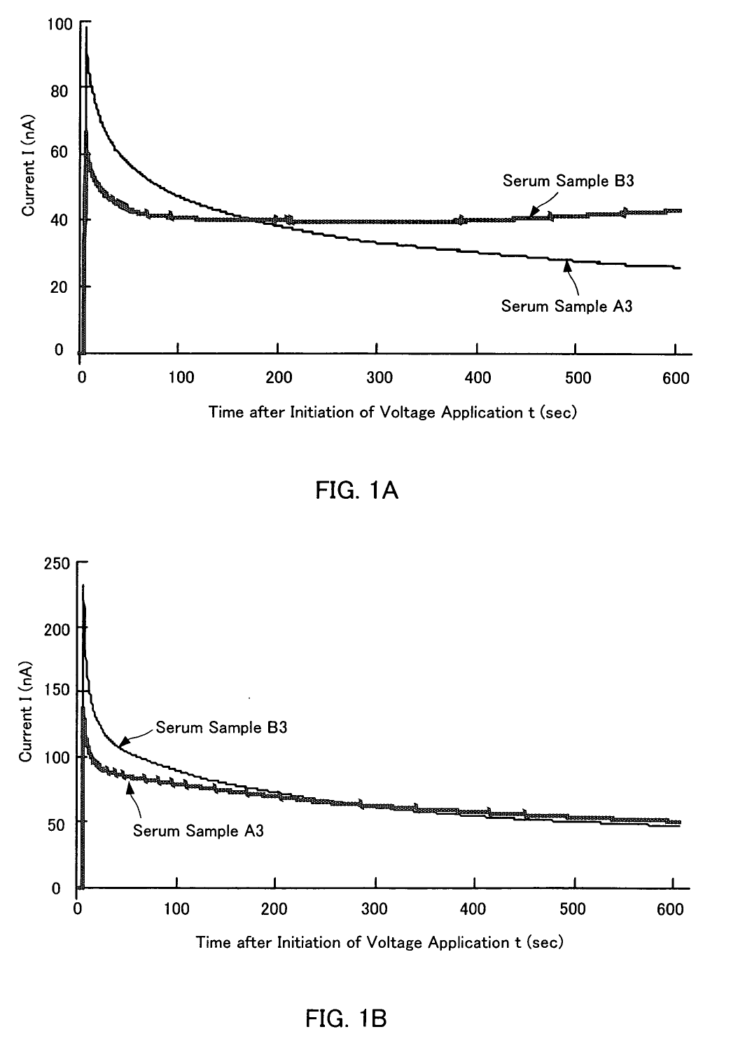

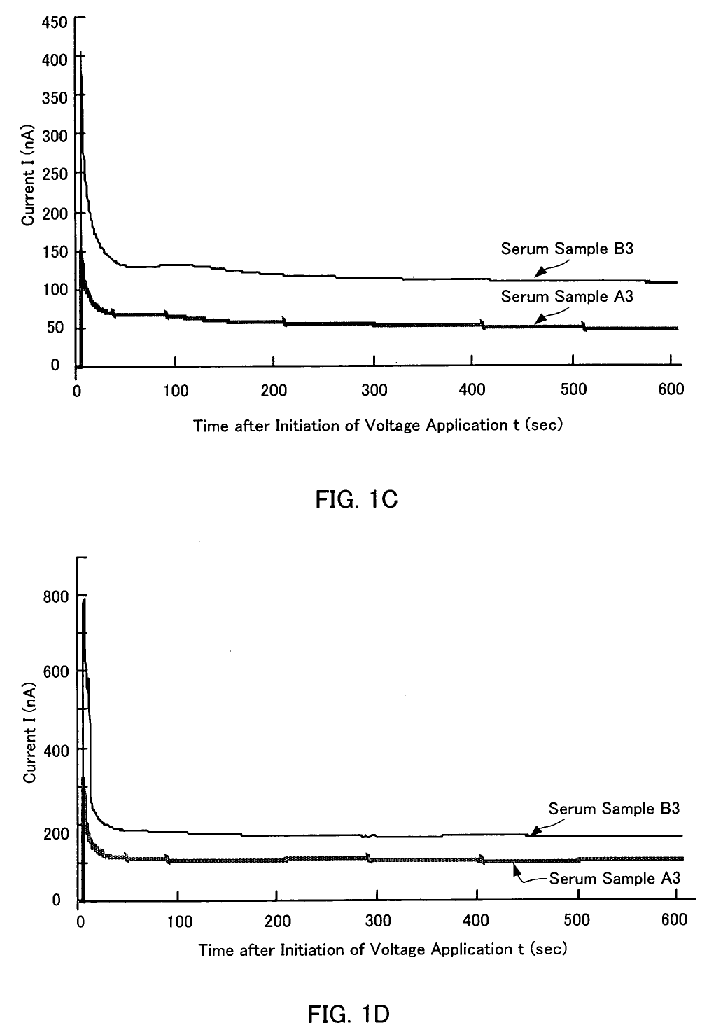

FIGS. 1A to 1D show the results of the constant-potential measurements in Examples 1 and 2 as well as Comparative Examples 1 and 2. FIG. 1A is a current curve monitored in Comparative Example 1. FIGS. 1B, 1C, and 1D are current curves monitored in Comparative Example 2, Example 1, and Example 2, respectively. In each graph, the point in time at which the voltage application (constant-potential measurement) was initiated is indicated as zero second.

In any of FIGS. 1A to 1D, a typical response current was observed in which after the peak current value was obtained immediately after the initiation of the constant-potential measurement, the current value decreased rapidly. In Comparative Examples 1 and 2, as shown in FIGS. 1A and 1B, responses in which the current value did not settle into a steady value were observed even after 600 seconds passed from the initiation of the measurement. On the other hand, a response in which the current value was brought into a steady state (settled into a steady value) was obtained between 180 seconds and 600 seconds after the initiation of the measurement in Example 1 as shown in FIG. 1C and was obtained between 80 seconds and 600 seconds after the initiation of the measurement in Example 2 as shown in FIG. 1D. Thus it was proved that a response current of a steady state was obtained between 180 seconds and 600 seconds, or between 80 seconds and 600 seconds in some cases, after the initiation of the constant-potential measurement, when the serum sample was filtered and the conditions to be employed for the filtering were controlled in a predetermined range.

Furthermore, in the serum sample A3, the activity of LDH contained therein was higher as compared to the serum sample B3 when the serum sample was not filtered, and therefore normally a large amount of response current should constantly be obtained. In such a serum sample A3, as shown in FIG. 1A, it was observed that the response current amount was smaller as compared to the serum sample B3 at 180 seconds and thereafter from the initiation of the measurement. Surprisingly, such a reverse phenomenon between the LDH activity and the amount of response current was observed even in the case where the filtering was carried out that removed molecules with molecular weights of 50000 or higher from the serum sample, i.e. the filtering was carried out under the conditions that allowed both albumin and immunoglobulin to be removed (Comparative Example 2, see FIG. 1B). This also proved that the current values corresponding to the magnitude of the LDH activity were obtained in the range between the point in time of the initiation of the constant-potential measurement and 600 seconds thereafter only when the serum sample was filtered and the conditions under which the filtering was carried out were controlled in a predetermined range.

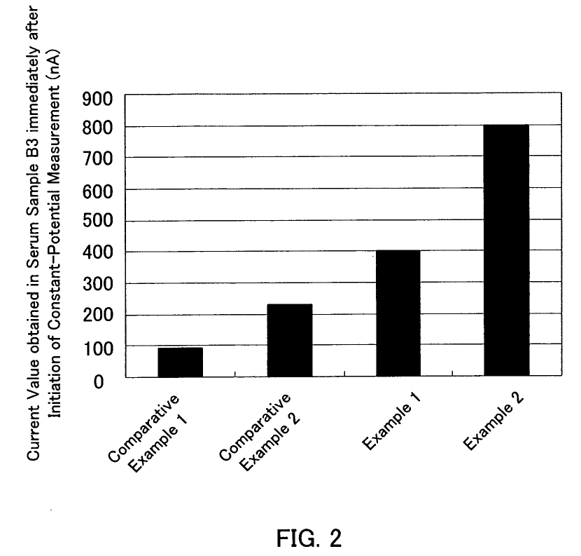

As shown in FIG. 2, in Comparative Examples 1 and 2, even when the peak value of response current was obtained, the signal obtained thereby was merely less than 250 nA, while in Examples 1 and 2, stronger signals were obtained, specifically, 400 nA in Example 1 and 800 nA in Example 2. Similarly in the case where the response current was in the steady state, considerably greater signal values were obtained in Examples 1 and 2 as compared to Comparative Examples 1 and 2. Thus, it was proved that when the conditions under which the serum sample was filtered were controlled in a predetermined range, it was possible to increase the magnitude of signals of response current considerably.

In electrochemical measurement of biomolecular activity, the mechanism that causes deterioration in measurement accuracy and an impediment to shortening the measurement time is unclear in many respects. However, one of the factors is considered as follows. That is, proteins that exist in serum adsorb to the surfaces of the measurement electrodes and thereby reduce the effective areas of the measurement electrodes. Since at least 100 kinds of proteins exist in blood, it is not easy to specify the responsible protein that causes the deterioration in measurement accuracy. However, according to the general common knowledge in the technical field concerned, albumin and various types of immunoglobulins (alpha 1, alpha2, beta, gamma, etc.) are predicted as the candidates. This is because such proteins each have a high mass ratio in serum and have a dominant effect on variations in the total amount of proteins in serum.

However, as is apparent from the result of Comparative Example 2 described above, contrary to such a prediction according to the general common knowledge in the technical field concerned, it was proved that surprisingly, even if filtering for removing molecules with molecular weights of 50000 or higher, i.e. filtering that allowed both albumin and immunoglobulin to be removed was carried out, the response current did not reach the steady state within 600 seconds after the initiation of the constant-potential measurement and the response current that reflected the magnitude of the LDH activity was not obtained. As described above, at the present stage, it is not clear why it is only after the removal of molecules whose molecular weights are 30000 or higher that such an excellent effect can be obtained.

### Example 3

Example 3 is an experimental example. In this example, a plurality of serum samples that were different in LDH activity from each other were subjected to constant-potential measurement carried out through the step 1 and step 2 described below. Then currents generated 180 seconds and 300 seconds after the initiation of voltage application were measured. Thereafter, the effect of an intrinsic redox substance that might be contained in the serum sample was corrected.

[Step 1]

First, five kinds of serum samples a to ξ were prepared that were different in LDH activity from each other. The serum sample α is the above-mentioned serum sample A. The serum samples β, γ, and ε are serum samples containing 605 U/L, 905 U/L, and 1205 U/L of LDH, each of which was prepared by artificially adding LDH (Product Number: 107085, manufactured by Roche Diagnostics, Inc.) to the serum sample A. The serum sample ξ is the serum sample B described above.

In parallel with this, a reaction solution (77.5 μL) was prepared that contained 50 mM of Tris-HCl buffer (pH 8.5), 1 mM of nicotinamide adenine dinucleotide (NAD), and 1 mM of potassium ferricyanide. Thereafter, it was incubated at 30° C. for three minutes. Five of these reaction solutions were prepared and were used as reaction solutions α to ξ.

Then 20 μL of 100-mM lithium lactate, 2.5 μL of 1000-U/L diaphorase, and 100 μL of each of the serum samples α to ξ were added to each of the reaction solutions α to ξ; They were incubated at room temperature (25° C.) for two minutes. Thereafter, each of them was filtered through a centrifugal filter (Amicon 10,000, manufactured by Amicon Inc.) that was able to remove molecules whose molecular weights were 10000 or higher. Hereinafter, the respective samples obtained after the filtering were referred to as measurement samples α to ξ. In this case, the filtering was carried out by the centrifugal process in which a centrifugal force of 12000G was applied for three minutes.

Then 40 μL of each of the above-mentioned measurement samples a to ξ was measured out and then was fed into a first electrode bath used for the constant-potential measurement that was provided with a working electrode, a counter electrode, and a reference electrode. Thereafter, a voltage of 400 mV with reference to a silver/silver chloride electrode was applied thereto and thereby current generated in each measurement sample was monitored after 180 seconds and 300 seconds from the initiation of the voltage application. This constant-potential measurement was carried out using a carbon electrode for each of the working electrode and the counter electrode and the silver/silver chloride electrode for the reference electrode.

[Step 2]

After 77.5 μL of 50-mM Tris-HCl buffer (pH 8.5) was incubated at 30° C. for three minutes, 22.5 μL of pure water (milliQ water) and 100 μL of each of the above-mentioned serum samples α to ξ were added thereto. Then they were allowed to stand still at room temperature (25° C.) for two minutes. Thereafter, each of them was filtered through the above-mentioned centrifugal filter (Amicon 10,000, manufactured by Amicon Inc.). Hereinafter, the respective samples obtained after the filtering are referred to as control samples α to ξ. In this case, the filtering was carried out by the centrifugal process in which a centrifugal force of 12000G was applied for three minutes.

Then 40 μL of each of the above-mentioned control samples α to ξ was measured out and then was fed into a second electrode bath used for the constant-potential measurement that was provided with a working electrode, a counter electrode, and a reference electrode. Thereafter, a voltage of 400 mV with reference to a silver/silver chloride electrode was applied thereto and thereby current generated in each sample was monitored after 180 seconds and 300 seconds from the initiation of the voltage application. This constant-potential measurement was carried out using a carbon electrode for each of the working electrode and the counter electrode and the silver/silver chloride electrode for the reference electrode.

The current value obtained from the control sample α (Step 2) was subtracted from the current value obtained from the measurement sample α (Step 1). Thus, the current value was calculated in which the effect of the reduced electron mediator that was present in the serum sample α was corrected.

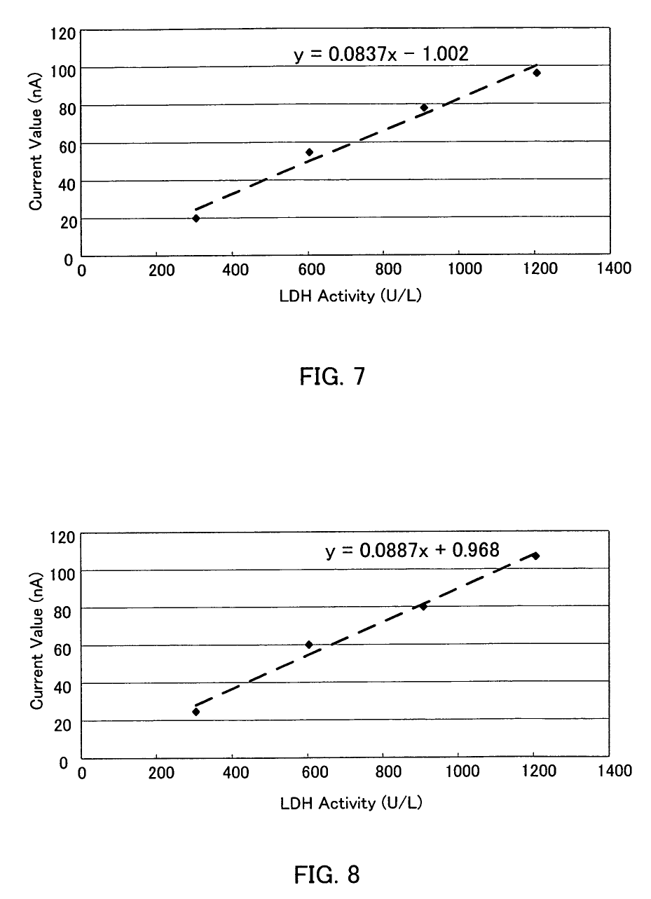

FIGS. 7 and 8 show the results obtained from the measurement samples α, β, γ, and ε. FIGS. 7 and 8 are graphs (vertical axis: current value (nA), horizontal axis: LDH activity (U/L)) that were obtained by plotting the corrected current values obtained from the measurement samples α, β, γ, and ε after 180 seconds and 300 seconds from the initiation of the constant-potential measurement, respectively. The dotted line is an approximate straight line determined by the least-squares method from the plot in each graph. In FIG. 7, the slope was 0.0837 and the intercept was −1.002 while in FIG. 8, the slope was 0.0887 and the intercept was 0.968. Thus, it was proved that the current values reflecting the LDH activity in the serum samples with good linearity were obtained.

Furthermore, with respect to the results of the constant-potential measurement obtained from the measurement sample ξ, the corrected current values obtained after 180 seconds and 300 seconds were 64.1 nA and 69.3 nA, respectively. Each corrected current value was assigned to the corresponding approximate straight line and thereby the LDH activity of the measurement sample ξ was calculated. As a result, the value calculated from the current value obtained after 180 seconds was 777.8 U/L, while the value calculated from the current value obtained after 300 seconds was 770.4 U/L. Thus, the values calculated above were those that approximate to the true activity value (772 U/L).

The above-mentioned measurement sample ξ was different in the LDH activity from the measurement samples α to ε. In addition, other proteins contained in the measurement sample ξ had concentrations that were approximately double the concentrations in the measurement samples α to ε. The concentrations of the biomolecules in serum vary considerably between individuals. Even in the same individual, they vary considerably according to, for example, the health condition at that time. Hence, this has a possibility of deteriorating the accuracy in detecting the LDH activity. However, from the results obtained by the comparison between the measurement sample ξ and the measurement samples α to ε, it was proved that by removing molecules whose molecular weights were 10000 or higher and correcting the current value of the sample subjected to the measurement, response current that had good linearity and reflected the LDH activity was obtained without being affected by the contents of other proteins.

### Example 4

Embodiment 4 is an experimental example in which the constant-potential measurement was carried out in the same manner as in Example 3 except that the centrifugal filter used herein was a centrifugal filter (Amicon 30,000, manufactured by Amicon, Inc.) that was able to remove molecules whose molecular weights were 30000 or higher.

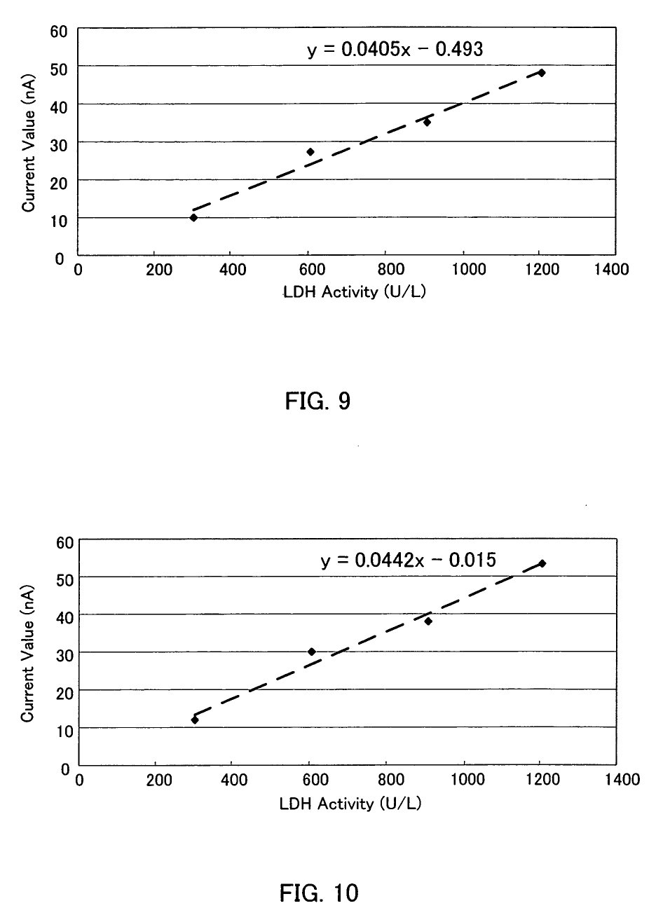

FIGS. 9 and 10 show the results obtained from the measurement samples α, β, γ, and ε. FIGS. 9 and 10 are graphs that were obtained by plotting corrected current values obtained from the measurement samples α, β, γ, and ε after 180 seconds and 300 seconds from the initiation of the constant-potential measurement, respectively. The dotted line is an approximate straight line determined by the least-squares method from the plot in each graph. In FIG. 9, the slope was 0.0405 and the intercept was −0.493, while in FIG. 10, the slope was 0.0442 and the intercept was −0.015. Thus, it was proved that the current values reflecting the LDH activity in the serum samples with good linearity were obtained.

Furthermore, with respect to the results of the constant-potential measurement obtained from the measurement sample ξ, the corrected current values obtained after 180 seconds and 300 seconds were 31.1 nA and 34.0 nA, respectively. Each corrected current value was assigned to the corresponding approximate straight line and thereby the LDH activity of the measurement sample ξ was calculated. As a result, the value calculated from the current value obtained after 180 seconds was 780.0 U/L, while the value calculated from the current value obtained after 300 seconds was 769.6 U/L. The values calculated above were those that approximate sufficiently to the true activity value (772 U/L), although they were not quite as approximate as in Example 3. Hence, it was proved that response current that had good linearity and sufficiently reflected the activity of LDH contained therein also was obtained by removing molecules whose molecular weights were 30000 or higher and detecting the amount of response current generated due to the internal electron mediator to correct the current value of the sample subjected to the measurement, regardless of the contents of proteins other than the LDH.

The present invention is applicable to improving the accuracy in detecting the LDH activity in the serum sample by the electrochemical technique and to shortening the time required for the detection. Furthermore, the present invention also is applicable to providing a measurement device that is suitable for the measurement described above.

What is claimed is:

1. A method of measuring lactate dehydrogenase activity in serum, the method comprising:
a first step of adding at least lactic acid, a coenzyme of a lactate dehydrogenase, and an electron mediator to a first serum sample that contains the lactate dehydrogenase to prepare a second serum sample;
a second step of removing molecules whose molecular weights are 30000 or higher from the second serum sample to prepare a third serum sample after the first step; and
a third step of measuring a value of current that is generated by applying voltage to the third serum sample after the second step.

2. The method according to claim 1, wherein molecules whose molecular weights are 10000 or higher also are removed in the second step.

3. The method according to claim 1, wherein in the third step, the value of current is measured between 180 seconds and 600 seconds after initiation of application of the voltage.

4. The method according to claim 2, wherein in the third step, the value of current is measured between 80 seconds and 600 seconds after initiation of application of the voltage.

5. The method according to claim 1, wherein the electron mediator is of an oxidized form and is at least one selected from the group consisting of hexacyanoferrate(III), 1,2-naphthoquinone-4-sulfonate, 2,6-dichlorophenol indophenol, dimethylbenzoquinone, 1-methoxy-5-methylphenazinium methyl sulfate, methylene blue, gallocyanine, thionine, phenazine methosulfate, and Meldola's blue.

6. The method according to claim 1, wherein the coenzyme is at least one selected from nicotinamide adenine dinucleotide and nicotinamide adenine dinucleotide phosphate.

7. The method according to claim 1, wherein in the first step, diaphorase further is added to the first serum sample.

8. The method according to claim 1, wherein in the third step, a voltage that is equal to or higher than oxidation potential of a reduced electron mediator that is contained in the third serum sample is applied.

9. The method according to claim 1, further comprising:
a fourth step of applying a voltage that is equal to the voltage to be applied in the third step, to a baseline measurement sample, to obtain a baseline current value that is a value of current generated in the baseline measurement sample, the baseline measurement sample being obtained by removing molecules whose molecular weights are 30000 or higher from the first serum sample; and
a fifth step of subtracting the baseline current value from the value of current measured in the third step.

10. The method according to claim 9, wherein the fourth step is carried out before the third step is completed.

11. The method according to claim 9, wherein the fourth step is carried out after the third step.

12. The method according to claim 9, wherein the baseline measurement sample is obtained by also removing molecules whose molecular weights are 10000 or higher from the first serum sample.

13. A device for measuring lactate dehydrogenase activity in serum, the device comprising:
a sample inlet for feeding a serum sample that contains lactate dehydrogenase;
a reaction bath for mixing the serum sample, lactic acid, a coenzyme of the lactate dehydrogenase, and an electron mediator, the reaction bath being in communication with the sample inlet;
a first electrode bath provided with electrodes; and
a molecule exclusion channel that allows the reaction bath and the first electrode bath to be in communication with each other,
wherein the molecule exclusion channel is provided with a member for removing molecules whose molecular weights are 30000 or higher from the serum sample that is discharged from the reaction bath.

14. The device according to claim 13, wherein the molecule exclusion channel is provided with a member for also removing molecules whose molecular weights are 10000 or higher from the serum sample that is discharged from the reaction bath.

15. The device according to claim 13, further comprising a second electrode bath provided with electrodes, and a second molecule exclusion channel that allows the sample inlet and the second electrode bath to be in communication with each other,
wherein the second molecule exclusion channel is provided with a member for removing molecules whose molecular weights are 30000 or higher from the serum sample that is discharged from the sample inlet.

16. The device according to claim 15, wherein the second molecule exclusion channel is provided with a member for also removing molecules whose molecular weights are 10000 or higher from the serum sample that is discharged from the sample inlet.
